(19) United States
(12) Patent Application Publication
(10) Pub. No.: US 2002/0161351 A1
(43) Pub. Date: Oct. 31, 2002

(54) Method and apparatus for treating acute myocardial infarction with selective hypothermic perfusion

(76) Inventors: Wilfred J. Samson, Saratoga, CA (US); John A. Macoviak, La Jolla, CA (US); Janine Robinson, Half Moon Bay, CA (US); Brady Esch, San Jose, CA (US)

Correspondence Address: Gunther Hanke, Fulwider Patton Lee & Utecht, P.O. Box 22615, Long Beach, CA, 90801-5615, US

(21) Appl. No.: 10/102,124

(22) Filed: Mar. 19, 2002

Related U.S. Application Data
(63) Continuation-in-part of application No. 09/384,467, filed on Aug. 27, 1999.
(60) Provisional application No. 60/098,727, filed on Sep. 1, 1998.

(51) Int. Cl. A61M 31/00; A61M 37/00; A61F 7/12

(52) U.S. Cl. 604/507; 604/6.13; 604/113

(57) ABSTRACT

An apparatus and method are described for therapeutic hypothermia of the heart by selective hypothermic perfusion of the myocardium through the patient's coronary arteries. The apparatus and method provide rapid cooling of the affected myocardium to achieve optimal myocardial salvage in a patient experiencing acute myocardial infarction. The therapeutic hypothermia system includes one or more selective coronary perfusion catheters and a fluid source for delivering a hypothermically-cooled physiologically-acceptable fluid, such as saline solution, oxygenated venous blood, autologously-oxygenated arterial blood and/or an oxygenated blood substitute. The system may also include one or more guidewires, subselective catheters and/or interventional catheters introduced through a lumen in the selective coronary perfusion catheter.

### CROSS REFERENCE TO OTHER APPLICATIONS

This application is a continuation-in-part of U.S. patent application Ser. No. 09/384,467, filed on Aug. 30, 1999, which claims the benefit of U.S. provisional application serial No. 60/098,727, filed on Sep. 1, 1998, the specifications of which are hereby incorporated in their entirety.

### FIELD OF THE INVENTION

The present invention relates generally to methods and devices for treatment of heart disease. More particularly, it relates to methods and devices for treating acute myocardial infarction with selective hypothermic perfusion.

### BACKGROUND OF THE INVENTION

Heart disease is the most common cause of death in the United States and in most countries of the western world. Coronary artery disease accounts for a large proportion of the deaths due to heart disease. Coronary artery disease is a form of atherosclerosis in which lipids, cholesterol and other materials deposit in the arterial walls gradually narrowing the arterial lumen, thereby depriving the myocardial tissue downstream from the narrowing of blood flow that supplies oxygen and other critical nutrients and electrolytes. These conditions can be further exacerbated by a blockage due to thrombosis, embolization or arterial dissection at the site of the stenosis. A severe reduction or blockage of blood flow can lead to ischemia, myocardial infarction and necrosis of the myocardial tissue.

Recent research has indicated that, during the acute stages of myocardial infarction, as much as half of the myocardial tissue at risk can be salvaged by hypothermic treatment of the ischemic area. It is theorized that hypothermia halts the progression of apoptosis or programmed cell death, which causes as much tissue necrosis as the ischemia that precipitated the myocardial infarction. To date, most attempts at hypothermic treatment for acute myocardial infarction have involved global hypothermia of the patient's entire body, for example using a blood heat exchanger inserted into the patient's vena cava. While this method has shown some efficacy in initial trials, it has a number of drawbacks. In particular, the need to cool the patient's entire body with the heat exchanger slows the process and delays the therapeutic effects of hypothermia. The more quickly the patient's heart can be cooled, the more myocardial tissue can be successfully salvaged. Global hypothermia has another disadvantage in that it can trigger shivering in the patient. A number of strategies have been devised to stop the patient from shivering, but these add to the complexity of the procedure and have additional risk associated with them. Shivering can be avoided altogether by induction of localized hypothermia of the heart or of the affected myocardium without global hypothermia. Localized hypothermia has the additional advantage that it can be achieved quickly because of the lower thermal mass of the heart compared to the patient's entire body. Rapid induction of therapeutic hypothermia gives the best chance of achieving the most myocardial salvage and therefore a better chance of a satisfactory recovery of the patient after acute myocardial infarction.

What would be desirable, but heretofore unavailable, is an apparatus and method for rapid induction of therapeutic hypothermia of the heart or of the affected myocardium in a patient experiencing acute myocardial infarction.

### SUMMARY OF THE INVENTION

In keeping with the foregoing discussion, the present invention provides an apparatus and method for induction of therapeutic hypothermia of the heart by selective hypothermic perfusion of the myocardium through the patient's coronary arteries. The apparatus and method provide rapid cooling of the affected myocardium to achieve optimal myocardial salvage in a patient experiencing acute myocardial infarction.

The apparatus takes the form of a therapeutic hypothermia system including at least one selective coronary perfusion catheter and a fluid source for delivering a hypothermically-cooled physiologically-acceptable fluid. The selective coronary perfusion catheter has an elongated catheter shaft configured for transluminal introduction via an arterial insertion site, such as a femoral, subclavian or brachial artery. The proximal end of the catheter shaft has a perfusion fitting configured for connecting to the fluid source. The distal end of the catheter shaft is preferably curved to selectively engage either the right or the left coronary artery. A perfusion lumen extends through the catheter shaft from the perfusion fitting at the proximal end to the distal end of the catheter shaft for delivering hypothermically-cooled physiologically-acceptable fluid from the fluid source to the patient's left or right coronary ostium. Optionally, two selective coronary perfusion catheters may be connected to the fluid source to allow simultaneous perfusion of both the right and left coronary arteries.

In one preferred embodiment, the selective coronary perfusion catheter includes one or more arch perfusion ports located on the exterior of the catheter shaft in the patient's aortic arch. Each arch perfusion port has a pressure-activated flow control valve for controlling fluid flow through the port(s). In addition, the selective coronary perfusion catheter may include an expandable flow control member located on the exterior of the catheter shaft in the patient's descending aorta. The expandable flow control member may be in the form of an inflatable balloon or a selectively-expandable external flow control valve.

The fluid source may take one of several possible forms. In one preferred embodiment, the fluid source includes an arterial cannula for withdrawing autologously-oxygenated blood from the patient, a heat exchanger for hypothermically cooling the withdrawn blood and a blood pump for pumping the blood through the heat exchanger and the selective coronary perfusion catheter into the patient's coronary artery. In another preferred embodiment, the fluid source includes a venous cannula for withdrawing venous blood from the patient, a heat exchanger for hypothermically cooling the venous blood, a blood oxygenator for oxygenating the blood and a blood pump for pumping the blood through the heat exchanger, the blood oxygenator and the selective coronary perfusion catheter into the patient's coronary artery. Alternatively, the fluid source may include a supply of another physiologically-acceptable fluid, such as saline solution or an oxygenated blood substitute, and a fluid pump or pressure source for pumping the fluid through the selective coronary perfusion catheter into the patient's coronary artery. The fluid source may also include a heat exchanger for hypothermically cooling the fluid or the fluid may be precooled, for example by storing the fluid in a refrigerator.

### BRIEF DESCRIPTION OF THE DRAWINGS

FIG. 1 is a cutaway view of a patient's thoracic aorta showing a distal end of a selective coronary perfusion catheter positioned for administering therapeutic hypothermia to the patient's myocardium.

FIG. 2 is an enlarged view of a portion of the selective coronary perfusion catheter of FIG. 1 showing an arch perfusion port with a pressure-activated flow control valve for controlling fluid flow through the port.

FIG. 3 is a cutaway view of the patient's abdominal aorta showing a proximal end of the selective coronary perfusion catheter of FIG. 1.

FIG. 4 is a schematic diagram of a therapeutic hypothermia system for delivering selective hypothermia to the patient's myocardium using a supply of blood or another physiologically-acceptable fluid.

FIG. 5 is a schematic diagram of a therapeutic hypothermia system for delivering selective hypothermia to the patient's myocardium using hypothermically-cooled autologously-oxygenated blood.

FIG. 6 is a schematic diagram of a therapeutic hypothermia system for delivering selective hypothermia to the patient's myocardium using hypothermically-cooled and oxygenated venous blood.

FIG. 7 shows a mechanically-actuated flow control valve for controlling fluid flow through the arch perfusion ports of the catheter shown in a closed position.

FIG. 8 shows the mechanically-actuated flow control valve of FIG. 7 shown in a closed position.

FIG. 9 shows an injection fitting with one-way valves for injection of a contrast medium and/or therapeutic agents through the lumen of the selective coronary perfusion catheter.

### DETAILED DESCRIPTION OF THE INVENTION

The present invention provides an apparatus and method for induction of therapeutic hypothermia of the heart by selective hypothermic perfusion of the myocardium through the patient's coronary arteries. The apparatus and method provide rapid cooling of the affected myocardium to achieve optimal myocardial salvage in a patient experiencing acute myocardial infarction.

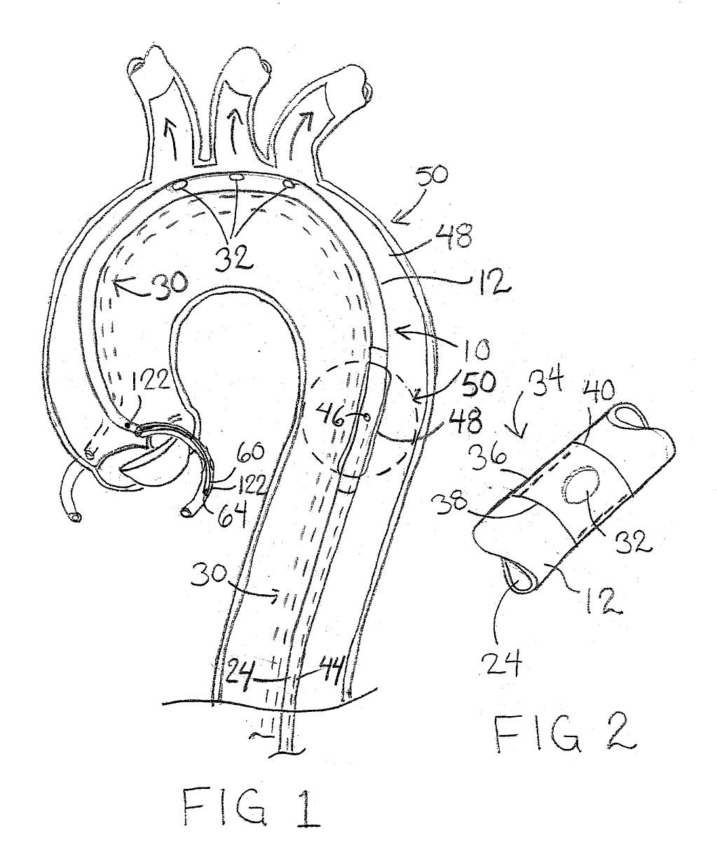

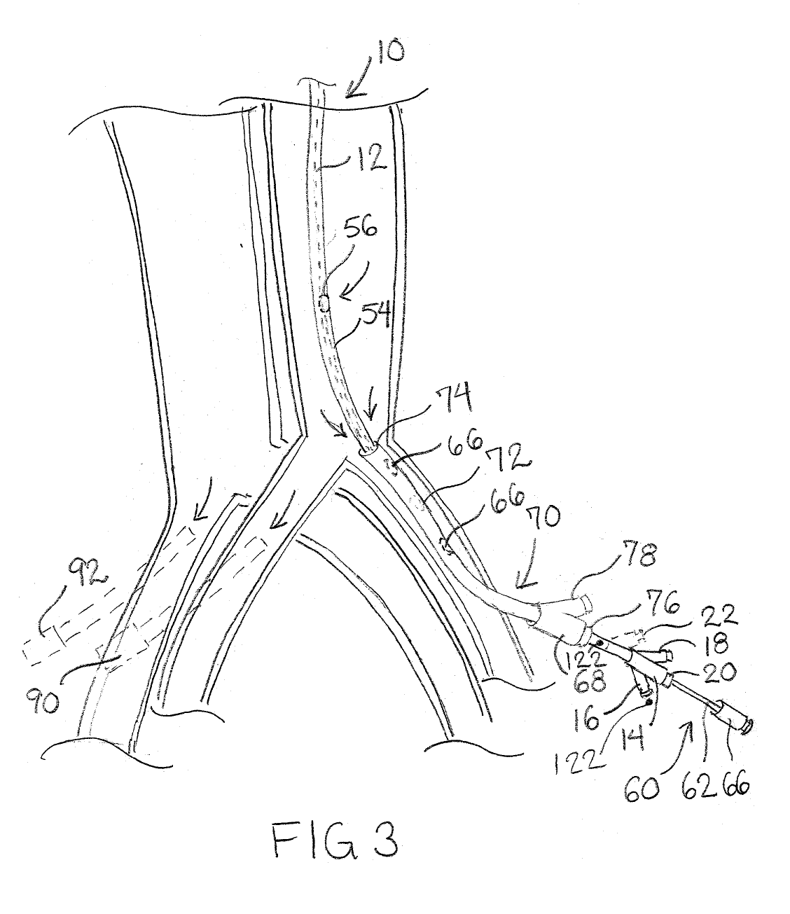

The apparatus takes the form of a therapeutic hypothermia system including at least one selective coronary perfusion catheter and a fluid source for delivering a hypothermically-cooled physiologically-acceptable fluid. FIG. 1 is a cutaway view of a patient's thoracic aorta showing a distal end of a selective coronary perfusion catheter 10 positioned for administering therapeutic hypothermia to the patient's myocardium. FIG. 3 is a cutaway view of the patient's abdominal aorta showing a proximal end of the selective coronary perfusion catheter 10 of FIG. 1. The selective coronary perfusion catheter 10 has an elongated catheter shaft 12 configured for transluminal introduction via an arterial insertion site, such as a femoral, subclavian or brachial artery. The catheter shaft 12 may be constructed of extruded polymeric tubing or, more preferably, of a fiber or wire braid-reinforced polymeric composite tubing. The catheter shaft 12 may have an outer diameter of approximately 1 to 3 mm and a length sufficient to extend from the arterial insertion site to the patient's aortic root. The length of the catheter shaft 12 may be from approximately 60 to 120 cm, depending on the arterial access site chosen. The distal end of the catheter shaft 12 is preferably curved to selectively engage either the right or the left coronary ostium or the catheter shaft 12 may be made with a multipurpose curve, which allows the operator to engage either coronary ostium. The proximal end of the catheter shaft 12 has a proximal fitting 14 configured for connecting to the fluid source. A perfusion lumen 24 extends through the catheter shaft 12 from a perfusion connector 16 on the proximal fitting 14 for delivering hypothermically-cooled physiologically-acceptable fluid from the fluid source to the patient's left or right coronary artery. Optionally, two selective coronary perfusion catheters 10, 30 may be connected to the fluid source to allow simultaneous perfusion of both the left and right coronary arteries.

In one preferred embodiment, the selective coronary perfusion catheter 10 includes one or more arch perfusion ports 32 located on the exterior of the catheter shaft 12 in the patient's ascending aorta and/or aortic arch. Preferably, the arch perfusion ports 32 are located near the superior aortic arch and directed upward toward the aortic arch vessels so that a majority of perfusate that exits the arch perfusion ports 32 enters the aortic arch vessels. Preferably, each arch perfusion port 32 has a pressure-activated flow control valve 34 for controlling fluid flow through the port(s) 32. FIG. 2 is an enlarged view of a portion of the catheter shaft 12 of the selective coronary perfusion catheter 10 of FIG. 1 showing an arch perfusion port 32 with a pressure-activated flow control valve 34 for controlling fluid flow through the port 32. The pressure-activated flow control valve 34 may be in the form of an elastomeric flap or sleeve 36 that covers the arch perfusion port 32. The distal end 38 of the elastomeric sleeve 36 is affixed to the catheter shaft 12, while the proximal end 40 of the elastomeric sleeve 36 is unattached. Preferably, the catheter 10 is constructed so that the elastomeric sleeve 36 is flush with the surface of the catheter shaft 12 when the pressure-activated flow control valve 34 is in a closed position. The elasticity of the elastomeric sleeve 36 is selected so that the pressure-activated flow control valve 34 remains closed until the backpressure within the perfusion lumen 24 reaches a predetermined level, then the flow control valve 34 opens to allow excess perfusate to exit the arch perfusion ports 32. Alternatively, the elastomeric flap or sleeve 36 may be constructed with one or more pores that remain closed until the backpressure within the perfusion lumen 24 reaches a predetermined level, whereupon the pore(s) open to allow perfusate to exit the arch perfusion ports 32.

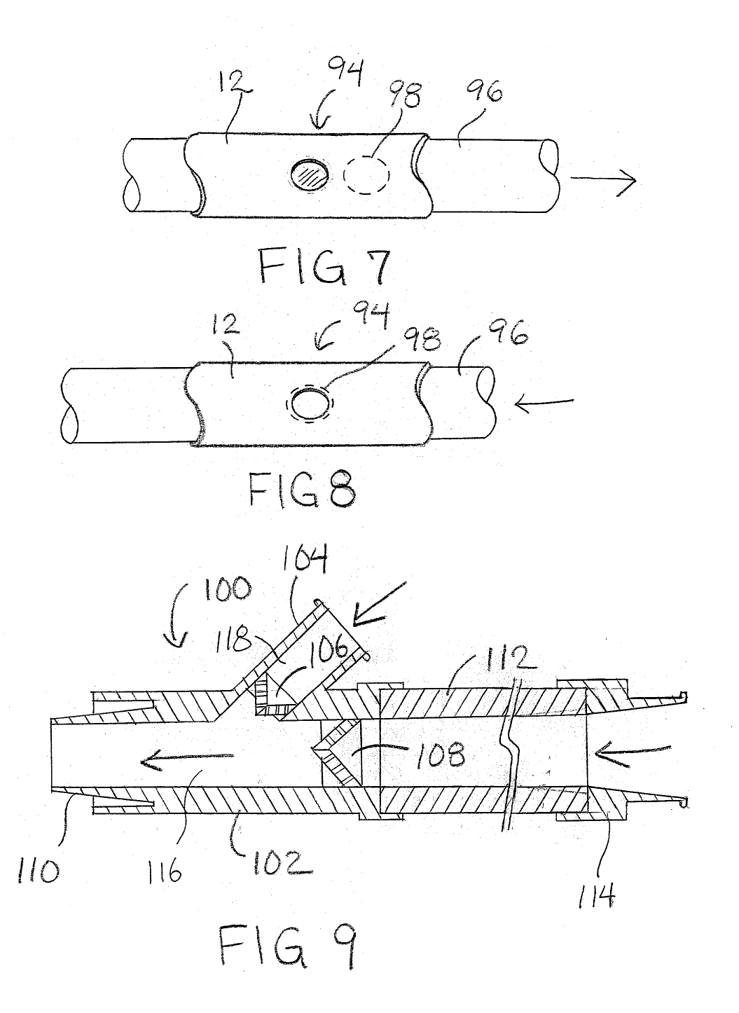

Alternatively, the catheter 10 may be constructed with mechanically-actuated flow control valves 94 for controlling fluid flow through the arch perfusion port(s) 32. In an exemplary embodiment shown in FIGS. 7 and 8, the mechanically-actuated flow control valve 94 includes a movable inner sleeve 96 with an aperture 98 through the wall of the inner sleeve 96. FIG. 7 shows the mechanically-actuated flow control valve 94 in a closed position with the wall of the inner sleeve 96 blocking flow through the arch perfusion port 32. To open the mechanically-actuated flow control valve 94, the inner sleeve 96 is rotated and/or moved axially to align the aperture 98 in the inner sleeve 96 with the arch perfusion port 32 in the catheter shaft 12, as shown in FIG. 8.

In addition, the selective coronary perfusion catheter 10 may include an expandable flow control member 50 located on the exterior of the catheter shaft 12 in the patient's descending aorta downstream of the aortic arch vessels. The expandable flow control member 50 may be in the form of an inflatable balloon 48, as shown, or in the form of a selectively-expandable external flow control valve. Selectively-expandable external flow control valves suitable for this application are described in U.S. Pat. No. 5,833,671, which is hereby incorporated by reference in its entirety. The interior of the inflatable balloon 48 is in fluid communication with a balloon inflation port 46 on the catheter shaft 12. A balloon inflation lumen 44 extends through the catheter shaft 12 from the balloon inflation port 46 to a balloon connector 18 on the proximal fitting 14.

The catheter shaft 12 is preferably made with a radiopaque construction, which facilitates viewing the catheter 10 by fluoroscopy. In addition, the catheter 10 may be constructed with one or more radiopaque and/or sonoreflective markers located along the catheter shaft 12 for visualizing the location of the distal tip of the catheter and the expandable flow control member 50 by fluoroscopy and/or ultrasonic imaging.

Optionally, the catheter 10 may be constructed with a blood withdrawal lumen 54 that extends through the catheter shaft 12 from one or more blood withdrawal ports 56 to a blood withdrawal connector 22 on the proximal fitting 14. Alternatively or in addition, the therapeutic hypothermia system may include an introducer sheath 70 for facilitating insertion of the selective coronary perfusion catheter 10 into the aorta from an arterial insertion site. Typically, the introducer sheath 70 will be constructed with a thin-walled tubular shaft 72 with a lumen 74 extending through it and a proximal fitting 68 with a hemostasis valve 76 or the like and a sidearm connector 78 for flushing the lumen 74 and/or for withdrawing arterial blood. Optionally, the introducer sheath 70 may have sideholes 66 in the tubular shaft 72 to facilitate blood entry into the lumen 74.

In addition, the proximal fitting 14 may be constructed with a hemostasis valve 20 or the like for introducing a guidewire and/or a subselective catheter 60 through the perfusion lumen 24 of the catheter 10. A subselective catheter 60 for use in the therapeutic hypothermia system may be configured as a flow guidewire, a subselective infusion catheter or an interventional catheter. For this application, the interior of the perfusion lumen 24 of the selective coronary perfusion catheter 10 will preferably have a lubricious or low friction surface to facilitate insertion of a catheter or guidewire through the catheter 10. When configured as a flow guidewire or subselective infusion catheter, the subselective catheter 60 will have an elongated shaft 62 with a lumen 64 extending through the shaft 62 from the proximal end to the distal end. Preferably, the exterior of the shaft 62 has a lubricious or low friction surface. A fitting 66 on the proximal end of the shaft 62 is configured for connecting the lumen 64 to a fluid source. The elongated shaft 62 is sized to fit through the perfusion lumen 24 of the selective coronary perfusion catheter 10 and may have an outer diameter of approximately 0.3 mm to 2 mm. The elongated shaft 62 has a length sufficient to extend through the perfusion lumen 24 of the selective coronary perfusion catheter 10 and advance distally beyond the distal end of the catheter shaft 12 into the patient's coronary artery. The flow guidewire or subselective catheter 60 can be used for administering subselective therapeutic hypothermia and/or for introducing an interventional catheter through the perfusion lumen 24 of the catheter 10. Subselective therapeutic hypothermia may also be administered through a lumen in the interventional catheter.

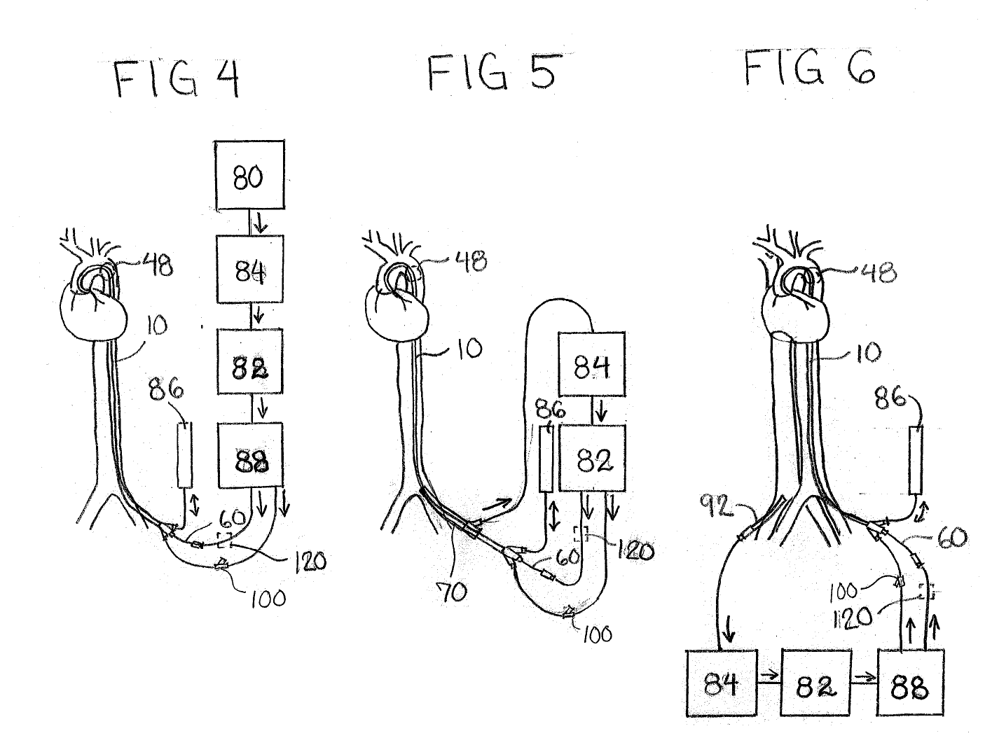

The fluid source for the therapeutic hypothermia system may take one of several possible forms. FIG. 4 is a schematic diagram of a therapeutic hypothermia system for delivering selective hypothermia to the patient's myocardium that includes a fluid supply reservoir 80 containing a physiologically-acceptable fluid and a fluid pump 84 (or other pressure source, for example an intravenous reservoir pressurization cuff) for pumping the fluid through the selective coronary perfusion catheter(s) 10 and/or the subselective catheter 60 into the patient's coronary artery or arteries. The fluid supply reservoir 80 may contain blood, saline solution, an oxygenated blood substitute or another physiologically-acceptable fluid.

Optionally, the therapeutic hypothermia system may include a heat exchanger 82 for hypothermically cooling the fluid from the fluid supply reservoir 80 before it enters the patient. Otherwise, the fluid may be precooled, for example by storing the fluid supply reservoir 80 in a refrigerator. This serves to simplify the therapeutic hypothermia system, which may save setup time in an emergency situation when the patient is in acute myocardial infarction. The therapeutic hypothermia system may also be prefilled with physiologically-acceptable fluid to facilitate setup in an emergency situation.

Optionally, the therapeutic hypothermia system may also include an oxygenator 88 for oxygenating the fluid from the fluid supply reservoir 80 before it enters the patient, such as when unoxygenated blood or an unoxygenated blood substitute are used. The use of a preoxygenated blood substitute, such as THEROX or PERFLUBRON, obviates the need for the oxygenator 88 and simplifies the system for faster setup in emergency situations.

When the selective coronary perfusion catheter 10 is constructed with an inflatable balloon 48 as a flow control member, the system will preferably include an inflation/deflation device 86 for inflating and deflating the balloon 48. Optionally, the inflation/deflation device 86 may include means for synchronizing the inflation and deflation of the balloon 48 with the patient's heartbeat.

FIG. 5 is a schematic diagram of a therapeutic hypothermia system for delivering selective hypothermia to the patient's myocardium using hypothermically-cooled autologously-oxygenated blood. Autologously-oxygenated arterial blood is withdrawn from the patient, pressurized by a blood pump 84, hypothermically cooled with a heat exchanger 82 and returned to the patient through the selective coronary perfusion catheter(s) 10 and/or the subselective catheter 60 into the patient's coronary artery or arteries. The autologously-oxygenated arterial blood can be withdrawn from the patient through an introducer sheath 70 coaxial to the catheter 10, as shown in FIG. 5, and/or through a blood withdrawal lumen 54 within the catheter 10 or an arterial cannula 90, as shown in FIG. 2. An arterial cannula 90 can be placed in the contralateral or ipsilateral femoral artery and/or at another arterial access site. The use of autologously-oxygenated blood simplifies the system by eliminating the need for a blood oxygenator. In addition, the use of a coaxial introducer sheath 70 or a blood withdrawal lumen 54 within the catheter 10 simplifies the procedure and eliminates the need for making a second arterial puncture for placement of a separate arterial cannula 90. Simplifying the system and the procedure allows for faster setup and thus more rapid and effective therapy in emergency situations when the patient is in acute myocardial infarction.

If the selective coronary perfusion catheter 10 is constructed with an inflatable balloon 48 as a flow control member, the system will preferably include an inflation/deflation device 86 for inflating and deflating the balloon 48. Optionally, the inflation/deflation device 86 may include means for synchronizing the inflation and deflation of the balloon 48 with the patient's heartbeat.

FIG. 6 is a schematic diagram of a therapeutic hypothermia system for delivering selective hypothermia to the patient's myocardium using hypothermically-cooled and oxygenated venous blood. Venous blood is withdrawn from the patient through a venous cannula 92, pressurized by a blood pump 84, hypothermically cooled with a heat exchanger 82, oxygenated by a blood oxygenator 88 and returned to the patient through the selective coronary perfusion catheter(s) 10 and/or the subselective catheter 60 into the patient's coronary artery or arteries. The venous cannula 92 can be placed in the contralateral or ipsilateral femoral vein and/or at another venous access site.

As in the previous examples, when the selective coronary perfusion catheter 10 is constructed with an inflatable balloon 48 as a flow control member, the system will preferably include an inflation/deflation device 86 for inflating and deflating the balloon 48. Optionally, the inflation/deflation device 86 may include means for synchronizing the inflation and deflation of the balloon 48 with the patient's heartbeat.

FIG. 9 shows an injection fitting 100 that may be utilized as part of the therapeutic hypothermia system. The injection fitting 100 has a main body 102 with a main channel 116 running through it and a male luer lock, barb connector or the like 110 at the distal end of the main channel 116 and a female luer lock, barb connector or the like 114 at the proximal end of the main channel 116. A side branch 104 with a female luer lock connector or the like 110 has a side branch channel 118 that connects to the main channel 116. A first one-way check valve 108 is positioned in the main channel 116 proximal to the takeoff of the side branch 104. The first one-way check valve 108 is configured to allow fluid to flow in the distal direction through the main channel 116 and to prevent flow in the proximal direction in the main channel 116. A second one-way check valve 106 is positioned in the side branch channel 118. The second one-way check valve 106 is configured to allow fluid to flow in the distal direction through side branch channel 118 into the main channel 116 and to prevent flow in the proximal direction in the side branch channel 118. Optionally, the injection fitting 100 may include an elastomeric extension tube 112 connecting the main body 102 with the female luer lock 114 on the proximal end of the main channel 116. The elastomeric extension tube 112 can expand to serve as a fluid accumulator for perfusate in the main channel 116 when fluid is injected through the side branch 104 of the injection fitting 100.

Optionally, the injection fitting 100 may be connected in series with the perfusion connector 16 on the proximal fitting 14 of the selective coronary perfusion catheter 100, as shown in FIGS. 4, 5 and 6. The injection fitting 100 facilitates injection of a radiopaque contrast medium, therapeutic agents and/or other fluids through the perfusion lumen 24 of the selective coronary perfusion catheter 10 via the side branch 104 without interrupting the flow of perfusate through the main channel 116.

Higher injection pressures may be needed for perfusing fluids at adequate therapeutic flow rates through a small-diameter flow guidewire or subselective catheter 60 compared to the perfusion pressure needed for the selective coronary perfusion catheter 100. To compensate for this, an optional second blood flow pump 120 may be connected in series to boost perfusion pressure through the flow guidewire or subselective catheter 60, as shown in FIGS. 4, 5 and 6.

It should be noted that the foregoing examples are provided as general guidelines for configuring the therapeutic hypothermia system. Some variation in the configuration and the order of the components in the fluid flow circuits may be acceptable. In addition, some or all of the components of the system may be combined to create a compact, integrated therapeutic hypothermia system.

The method of the present invention can be used in an emergency situation for treating a patient in acute myocardial infarction with therapeutic hypothermia or it can be used electively to create a protective hypothernic environment for the patient's myocardium prior to, during or after performing a catheter-based intervention. To begin, one or more of the patient's coronary arteries is selectively catheterized using the selective coronary perfusion catheter 10 as described above. A diagnostic angiogram can be performed by injecting radiopaque dye through the selective coronary perfusion catheter 10 to determine the location and severity of any lesions in the coronary arteries. Meanwhile, the fluid source is set up according to one of the examples shown in FIGS. 4, 5 and 6. The proximal fitting 14 of the catheter 10 is connected to the fluid source and therapeutic infusion of hypothermically-cooled fluid is begun. For emergency situations, the system setup, catheterization and initiation of therapeutic hypothermia should be done as rapidly as possible in order to effectively salvage as much of the myocardium as possible.

In addition to the above, a flow guidewire or subselective catheter 60 may be introduced through the selective catheter 10 and advanced into the patient's coronary artery. Depending on the location and severity of the coronary lesion, the subselective catheter 60 may be advanced across the lesion for therapeutic infusion of hypothermically-cooled fluid to the threatened myocardium downstream of the lesion. Alternatively or in addition, a therapeutic catheter such as an angioplasty, atherectomy or stent delivery catheter may be introduced through the selective catheter 10 and advanced into the patient's coronary artery for treating one or more of the coronary lesions. The hypothermic environment created by the therapeutic hypothermia system protects the patient's myocardium, reducing the risk of any catheter-based intervention and reducing the likelihood of reperfusion injury to the myocardium downstream of the lesion.

Therapeutic agents, such as thrombolytic agents and pharmacological agents for reducing reperfusion injury, can be administered through the selective coronary perfusion catheter(s) 10 and/or the subselective catheter 60 into the patient's coronary artery or arteries. Optionally, a pharmacological agent effective to slow the patient's heartbeat without arresting the heart can be administered through the selective coronary perfusion catheter(s) 10 to reduce the metabolic demand of the myocardium, which may result in less ischemic damage and more effective myocardial salvage.

Preferably, the therapeutic hypothermia system cools the patient's myocardium to a temperature of approximately 28 to 36 C., more preferably to a temperature of approximately 32 to 35 C., to create a protective hypothermic environment without stopping the heart and without significant risk of nerve block or induced arrhythmias, which can be a consequence of more profound hypothermia. In one preferred method, an initial bolus of cold perfusate at a temperature of approximately 10 to 20 C. may be infused to rapidly initiate therapeutic hypothermia, followed by steady infusion of perfusate at a temperature closer to the target temperature range of approximately 28 to 36 C. or 32 to 35 C., depending on the clinical protocol that is selected. In another preferred method, a low flow rate of cold perfusate, for example saline solution, at a temperature of approximately 10 to 20 C. may be added to the patient's native coronary blood flow to achieve an average temperature in the desired therapeutic temperature range.

Temperature feedback may be used to control the temperature and/or flow rate of the hypothermically-cooled fluid to achieve optimum therapeutic effect. Optionally, temperature sensors 122 may be incorporated into the therapeutic hypothermia system in the heat exchanger 82, in the proximal and/or distal end of the selective coronary perfusion catheter 10 and/or in the guidewire or subselective catheter 60, as shown in FIGS. 1 and 2. A feedback signal from the temperature sensor(s) 122 will be used to adjust the temperature and/or flow rate of the perfusate to achieve the desired tissue temperatures for effective therapeutic hypothermia.

When the backpressure within the perfusion lumen 24 of the selective coronary perfusion catheter 10 reaches a predetermined level, the flow control valve(s) 34 open to allow excess perfusate to exit the arch perfusion port(s) 32. In alternative embodiments, the mechanically-actuated flow control valve(s) 94 may be selectively opened to allow flow through the arch perfusion port(s) 32. The cold perfusate exiting the arch perfusion ports 32 mixes with the blood in the ascending aorta and aortic arch. Because the arch perfusion ports 32 are located near and directed upward toward the superior aortic arch, a majority of cold perfusate that exits the arch perfusion ports 32 enters the aortic arch vessels. This mechanism has two benefits. It prevents any potential damage from overperfusion of the coronary arteries and it provides a measure of hypothermic protection to the brain by way of the arch vessels.

If desired, the expandable flow control member 50 may be expanded to resist or to occlude blood flow in the patient's descending aorta downstream of the aortic arch vessels. This provides a greater proportion of the patient's cardiac output to the brain and the coronary arteries without significantly compromising the organ systems downstream of the aortic arch, which are much more resistant to ischemic damage.

Optionally, the expandable flow control member 50 may be synchronized with the patient's heartbeat. For example, when the expandable flow control member 50 is in the form of an inflatable balloon 48, the inflation/deflation device 86 may be constructed with means for synchronizing the inflation and deflation of the balloon 48 with the patient's heartbeat. The inflation/deflation device 86 may be synchronized using the patient's EKG signal or any other indicator of the cardiac cycle. The inflatable balloon 48 may be synchronized to inflate during diastole (counterpulsation), which will result in increased blood flow to the patient's coronary arteries. Alternatively, the inflatable balloon 48 may be synchronized to inflate during systole, which will result in increased blood flow to the patient's coronary arteries.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

We claim:

1. A method for treating acute myocardial infarction in a patient comprising:
introducing a catheter having a proximal end and a distal end and a lumen extending through the catheter from the proximal end to the distal end into the patient's aorta;
engaging at least one coronary artery of the patient with the distal end of the catheter; and
perfusing the coronary artery of the patient through the lumen of the catheter with a hypothermically-cooled physiologically-acceptable fluid to hypothermically cool a portion of the patient's myocardium without arresting the patient's heart.

2. The method of claim 1, wherein the catheter is introduced into the patient's aorta via a peripheral artery access site.

3. The method of claim 1, wherein the method further comprises positioning the distal end of the catheter at an ostium of the patient's coronary artery and introducing a subselective catheter through the lumen of the catheter and advancing a distal end of the subselective catheter to a point distal to a stenosis in the patient's coronary artery and perfusing the coronary artery distal to the stenosis through a lumen in the subselective catheter with the hypothermically-cooled physiologically-acceptable fluid.

4. The method of claim 3, wherein the method further comprises introducing an interventional catheter over the subselective catheter positioned within the lumen of the catheter and performing a catheter-based intervention on the patient's coronary artery.

5. The method of claim 1, wherein the method further comprises introducing a second catheter into the patient's aorta, engaging a second coronary artery of the patient with a distal end of the second catheter and perfusing the second coronary artery through a lumen in the second catheter with the hypothermically-cooled physiologically-acceptable fluid.

6. The method of claim 1, wherein the hypothermically-cooled physiologically-acceptable fluid is at a temperature of approximately 28 to 36 C.

7. The method of claim 1, wherein the hypothermically-cooled physiologically-acceptable fluid is at a temperature of approximately 32 to 35 C.

8. The method of claim 1, wherein the hypothermically-cooled physiologically-acceptable fluid includes a pharmacological agent effective to slow the patient's heartbeat without arresting the heart.

9. The method of claim 1, wherein at least a portion of the patient's myocardium is cooled to a temperature of approximately 28 to 36 C.

10. The method of claim 1, wherein at least a portion of the patient's myocardium is cooled to a temperature of approximately 32 to 35 C.

11. The method of claim 1, wherein the hypothermically-cooled physiologically-acceptable fluid comprises hypothermically-cooled saline solution.

12. The method of claim 1, wherein the hypothermically-cooled physiologically-acceptable fluid comprises a hypothermically-cooled oxygenated blood substitute.

13. The method of claim 1, wherein the hypothernically-cooled physiologically-acceptable fluid comprises hypothermically-cooled autologously-oxygenated blood.

14. The method of claim 1, wherein the method further comprises withdrawing autologously-oxygenated blood from an artery of the patient, hypothermically cooling the autologously-oxygenated blood and returning the hypothermically-cooled autologously-oxygenated blood to the patient through the lumen of the catheter.

15. The method of claim 1, wherein the autologously-oxygenated blood is withdrawn from the patient's artery through a second lumen within the catheter.

16. The method of claim 1, wherein the autologously-oxygenated blood is withdrawn from the patient's artery through a lumen within a sheath surrounding the catheter.

17. The method of claim 1, wherein the method further comprises withdrawing venous blood from a vein of the patient, oxygenating the blood, hypothermically cooling the oxygenated blood and returning the hypothermically-cooled oxygenated blood to the patient through the lumen of the catheter.

18. The method of claim 1, wherein the method further comprises perfusing the patient's arch vessels with the hypothermically-cooled physiologically-acceptable fluid through at least one arch perfusion port in fluid communication with the lumen of the catheter.

19. The method of claim 18, wherein the catheter comprises at least one pressure release valve positioned to control fluid flow through the at least one arch perfusion port, and wherein the method further comprises opening the at least one pressure release valve to perfuse the patient's arch vessels with the hypothermically-cooled physiologically-acceptable fluid through the at least one arch perfusion port when backpressure in the lumen of the catheter reaches a predetermined level.

20. The method of claim 1, wherein the method further comprises expanding a selectively-deployable blood flow control member mounted on an exterior of the catheter to resist blood within the patient's descending aorta downstream of the patient's arch vessels.

21. The method of claim 1, wherein the method further comprises expanding a selectively-deployable blood flow control member mounted on an exterior of the catheter in synchrony with the patient's heartbeat to resist blood within the patient's descending aorta downstream of the patient's arch vessels.

22. The method of claim 1, wherein the method further comprises performing a catheter-based intervention on at least one of the patient's coronary arteries after hypothermically cooling the patient's myocardium.

23. The method of claim 1, wherein the method further comprises introducing an interventional catheter through the lumen of the catheter and performing a catheter-based intervention on at least one of the patient's coronary arteries after hypothermically cooling the patient's myocardium.

24. Apparatus for treating acute myocardial infarction in a patient comprising:
a catheter having a proximal end and a distal end and a lumen extending through the catheter from the proximal end to the distal end, the catheter having a length sufficient to extend from a peripheral artery access site into the patient's aortic root, the distal end of the catheter being configured to engage a patient's coronary artery;
a source of hypothermically-cooled physiologically-acceptable fluid connected to the lumen at the proximal end of the catheter, the hypothermically-cooled physiologically-acceptable fluid having a temperature and a composition sufficient to hypothermically cool a portion of the patient's myocardium without arresting the patient's heart.

25. The apparatus of claim 24, wherein the hypothermically-cooled physiologically-acceptable fluid is at a temperature of approximately 28 to 36 C.

26. The apparatus of claim 24, wherein the hypothermically-cooled physiologically-acceptable fluid is at a temperature of approximately 32 to 35 C.

27. The apparatus of claim 24, wherein the hypothermically-cooled physiologically-acceptable fluid includes a pharmacological agent effective to slow the patient's heartbeat without arresting the heart.

28. The apparatus of claim 24, wherein the hypothermically-cooled physiologically-acceptable fluid comprises hypothermically-cooled saline solution.

29. The apparatus of claim 24, wherein the hypothermically-cooled physiologically-acceptable fluid comprises a hypothermically-cooled oxygenated blood substitute.

30. The apparatus of claim 24, wherein the hypothermically-cooled physiologically-acceptable fluid comprises hypothermically-cooled oxygenated blood.

31. The apparatus of claim 24, wherein the source of hypothermically-cooled physiologically-acceptable fluid comprises an arterial cannula for withdrawing autologously-oxygenated blood from an artery of the patient, a heat exchanger for hypothermically cooling the autologously-oxygenated blood and a pump for returning the hypothermically-cooled autologously-oxygenated blood to the patient through the lumen of the catheter.

32. The apparatus of claim 31, further comprising a subselective catheter having a proximal end and a distal end and a lumen extending through the subselective catheter from the proximal end to the distal end, the distal end of the subselective catheter being sized and configured to be introduced through the lumen of the catheter and advanced to a point distal to the distal end of the catheter, the lumen at the proximal end of the subselective catheter being connected to the source of hypothermically-cooled physiologically-acceptable fluid.

33. The apparatus of claim 31, further comprising an interventional catheter sized and configured for introduction through the lumen of the catheter.

34. The apparatus of claim 24, wherein the source of hypothermically-cooled physiologically-acceptable fluid comprises a second lumen within the catheter for withdrawing autologously-oxygenated blood from the aorta of the patient, a heat exchanger for hypothermically cooling the autologously-oxygenated blood and a pump for returning the hypothermically-cooled autologously-oxygenated blood to the patient through the lumen of the catheter.

35. The apparatus of claim 34, further comprising a subselective catheter having a proximal end and a distal end and a lumen extending through the subselective catheter from the proximal end to the distal end, the distal end of the subselective catheter being sized and configured to be introduced through the lumen of the catheter and advanced to a point distal to the distal end of the catheter, the lumen at the proximal end of the subselective catheter being connected to the source of hypothermically-cooled physiologically-acceptable fluid.

36. The apparatus of claim 34, further comprising an interventional catheter sized and configured for introduction through the lumen of the catheter.

37. The apparatus of claim 24, wherein the source of hypothermically-cooled physiologically-acceptable fluid comprises a lumen within a sheath surrounding the catheter for withdrawing autologously-oxygenated blood from an artery of the patient, a heat exchanger for hypothermically cooling the autologously-oxygenated blood and a pump for returning the hypothermically-cooled autologously-oxygenated blood to the patient through the lumen of the catheter.

38. The apparatus of claim 37, further comprising a subselective catheter having a proximal end and a distal end and a lumen extending through the subselective catheter from the proximal end to the distal end, the distal end of the subselective catheter being sized and configured to be introduced through the lumen of the catheter and advanced to a point distal to the distal end of the catheter, the lumen at the proximal end of the subselective catheter being connected to the source of hypothermically-cooled physiologically-acceptable fluid.

39. The apparatus of claim 37, further comprising an interventional catheter sized and configured for introduction through the lumen of the catheter.

40. The apparatus of claim 24, wherein the source of hypothermically-cooled physiologically-acceptable fluid comprises a venous cannula for withdrawing venous blood from a vein of the patient, a heat exchanger for hypothermically cooling the blood, a blood oxygenator for oxygenating the blood and a pump for returning the hypothermically-cooled oxygenated blood to the patient through the lumen of the catheter.

41. The apparatus of claim 40, further comprising a subselective catheter having a proximal end and a distal end and a lumen extending through the subselective catheter from the proximal end to the distal end, the distal end of the subselective catheter being sized and configured to be introduced through the lumen of the catheter and advanced to a point distal to the distal end of the catheter, the lumen at the proximal end of the subselective catheter being connected to the source of hypothermically-cooled physiologically-acceptable fluid.

42. The apparatus of claim 40, further comprising an interventional catheter sized and configured for introduction through the lumen of the catheter.

43. The apparatus of claim 24, wherein the catheter comprises at least one arch perfusion port in fluid communication with the lumen of the catheter.

44. The apparatus of claim 24, wherein the catheter comprises at least one arch perfusion port in fluid communication with the lumen of the catheter and at least one pressure release valve positioned to control fluid flow through the at least one arch perfusion port, and wherein the at least one pressure release valve is configured to allow fluid flow through the at least one arch perfusion port when backpressure in the lumen of the catheter reaches a predetermined level.

45. The apparatus of claim 24, further comprising a selectively-deployable expanding blood flow control member mounted on an exterior of the catheter to resist blood within the patient's descending aorta downstream of the patient's arch vessels.

46. The apparatus of claim 24, wherein the selectively-deployable expanding blood flow control member is an inflatable balloon.

47. The apparatus of claim 24, wherein the selectively-deployable expanding blood flow control member is a selectively-expandable external flow control valve.

48. The apparatus of claim 47, further comprising means for expanding the selectively-deployable expanding blood flow control member in synchrony with the patient's heartbeat to resist blood within the patient's descending aorta downstream of the patient's arch vessels.

49. The apparatus of claim 24, further comprising a second catheter having a proximal end and a distal end and a lumen extending through the second catheter from the proximal end to the distal end, the second catheter having a length sufficient to extend from a peripheral artery access site into the patient's aortic root, the distal end of the second catheter being configured to engage a second coronary artery of the patient, the lumen at the proximal end of the second catheter being connected to the source of hypothermically-cooled physiologically-acceptable fluid.

50. The apparatus of claim 24, further comprising a subselective catheter having a proximal end and a distal end and a lumen extending through the subselective catheter from the proximal end to the distal end, the distal end of the subselective catheter being sized and configured to be introduced through the lumen of the catheter and advanced to a point distal to the distal end of the catheter, the lumen at the proximal end of the subselective catheter being connected to the source of hypothermically-cooled physiologically-acceptable fluid.

51. The apparatus of claim 24, further comprising an interventional catheter sized and configured for introduction through the lumen of the catheter.

52. The apparatus of claim 24, further comprising a temperature sensor for measuring the temperature of hypothermically-cooled physiologically-acceptable fluid flowing through the lumen of the catheter.

53. The apparatus of claim 24, further comprising a temperature sensor for measuring the temperature of the physiologically-acceptable fluid flowing within a lumen of a coronary artery.
